(19) United States
(12) Patent Application Publication
(10) Pub. No.: US 2005/0089508 A1
(43) Pub. Date: Apr. 28, 2005

(54) Method of using GM-CSF to modulate food intake and regulate body weight

(76) Inventors: Jacquelyn Ann Reed, Cincinnati, OH (US); Deborah Joy Clegg, Cincinnati, OH (US); Kathleen Blake Smith, Cincinnati, OH (US); Randy John Seeley, Cincinnati, OH (US)

Correspondence Address: DINSMORE & SHOHL, LLP, 1900 CHEMED CENTER, 255 EAST FIFTH STREET, CINCINNATI, OH, 45202, US

(21) Appl. No.: 10/874,496

(22) Filed: Jun. 23, 2004

Related U.S. Application Data
(60) Provisional application No. 60/480,804, filed on Jun. 23, 2003.

(51) Int. Cl. A61K 38/17

(52) U.S. Cl. 424/85.1

(57) ABSTRACT

Methods of modulating metabolic activity and regulating body weight comprising administering to a subject an effective amount of an agent that acts via a GM-CSF signaling pathway, and methods of modulating metabolic activity and regulating body weight comprising manipulating a subject to intrinsically express an agent in an effective amount wherein the agent acts via a GM-CSF signaling pathway.

### RELATED APPLICATION

This application claims priority under U.S.C. § 119, of U.S. Provisional Application Ser. No. 60/480,804, filed Jun. 23, 2003.

### FIELD OF THE INVENTION

The present invention is directed toward methods for modulating metabolic activity and methods for regulating body weight. The methods employ Granulocyte-macrophage colony-stimulating factor, or agents which act via GM-CSF signaling pathways. More particularly, the methods may be used for treatment of both idiopathic and disease-related disorders of body weight.

### BACKGROUND OF THE INVENTION

The incidence of obesity, anorexia, and other body weight disorders is on the rise in the industrial world. The popular media has devoted enormous air and paper resources to reporting on the prevalence of obesity and the comorbidities of associated conditions. A multitrillion dollar diet and weight-loss industry has developed to address these disorders, and there are staggering costs in terms of medical dollars and loss of human potential due to shortened life spans and diminished quality of life. Less popularized, but similarly on the rise are the chachexia, or wasting disorders, which are characterized by decreased food intake and/or body weight related to underlying disease states such as AIDs, cancer and cancer treatments including chemotherapy.

Yet, obesity and other body weight disorders are not simple conditions of eating too much or too little. Metabolic activity, food intake, and, ultimately, body weight, are modulated and regulated according to exquisite physiochemical control mechanisms. Many of the currently available pharmaceutical agents targeted for treatment of body weight disorders are no more than substitutes for behavioral manipulations, and don't operate at the level of the control cues themselves. Hence, these therapies suffer from lack of efficacy and high rates of recidivism in many individuals. There is a substantial need for pharmacological therapies that modulate food intake and regulate body weight more effectively.

In the case of individuals suffering from chachexia (wasting disorders), there is the additional complication that treatment of the underlying disease may be unknowingly and unintentionally exacerbating the wasting by, for example, suppressing appetite. Hence, there is need for treatments which exploit or inhibit the secondary impacts on food intake and/or body weight, according to the need of the individual. Clearly there is a need for methods of modulating food intake and regulating body weight which take into consideration such secondary effects.

Given the wide clinical use of GM-CSF in the treatment of individuals with diseases associated with wasting conditions, methods which employ agents which modulate metabolic activity such as food intake and regulate body weight by acting on the GM-CSF signaling pathway would be highly desirable. In addition, there is a need for methods of treating idiopathic, as well as disease-related disorders of body weight.

### SUMMARY OF THE INVENTION

Accordingly, it is an object of this invention to provide methods for modulating metabolic activity, including food intake, and for regulating body weight.

One embodiment of the invention is directed to methods for modulating metabolic activity comprising administering to a subject GM-CSF or an agent that acts via a GM-CSF signaling pathway.

Another aspect of the invention is directed to methods for modulating metabolic activity comprising manipulating a subject to express an agent in an amount effective to modulate metabolic activity wherein the agent comprises GM-CSF or acts via a GM-CSF signaling pathway.

A further embodiment of the invention is directed to methods for regulating body weight comprising administering to a subject GM-CSF or an agent that acts via a GM-CSF signaling pathway.

Another aspect of the invention is directed to methods of regulating body weight comprising manipulating a subject to express an agent in an amount effective to regulate body weight wherein the agent comprises GM-CSF or acts via a GM-CSF signaling pathway.

An additional aspect of the invention is directed to methods for treatment of both idiopathic and disease-related body weight disorders comprising administering an agent that acts via a GM-CSF signaling pathway in effective amounts.

Another embodiment is directed to methods for identifying compounds which would be candidates for regulating metabolic activity or body weight in a species of therapeutic interest. The methods comprise bringing the test compound into contact with the GM-CSF receptor, determining whether that compound binds to the GM-CSF receptor, selecting out those compounds which do bind and further determining whether that compound modulates metabolic activity or body weight in a model system capable of evaluating metabolic activity or body weight. Finally, those test compounds which modulate metabolic activity or body weight in a model system are identified as candidate compounds for regulating metabolic activity or body weight in the species of therapeutic interest.

A final embodiment of the invention is directed to methods for identifying compounds which would be candidates for regulating metabolic activity or body weight in a species of therapeutic interest which comprise bringing test compounds into contact with a cell expressing a functional GM-CSF receptor, determining whether the compound activates the GM-CSF receptor, selecting the compounds which do activate the GM-CSF receptor and further determining whether those test compounds modulate metabolic activity or body weight in a model system that is capable of evaluating metabolic activity or body weight, and identifying those test compounds for regulating metabolic activity or body weight in a species of therapeutic interest.

The present methods are advantageous for modulating metabolic activity and regulating body weight. Additional embodiments, objects and advantages of the invention will become more fully apparent in view of the following detailed description.

### DETAILED DESCRIPTION

Granulocyte-macrophage colony-stimulating factor (GM-CSF) is a proinflammatory cytokine primarily expressed in bone marrow cells, endothelial cells, and cells of myeloctyic origin. It has been found to be critical for normal pulmonary surfactant homeostasis. GM-CSF signaling pathways, i.e. pathways which comprise receptors which are excited or inhibited by GM-CSF, GM-CSF agonists, or in response to changes in GM-CSF concentrations, are found in both the peripheral and central nervous systems. Elevated plasma GM-CSF is associated with inflammation and HIV, and is secreted by various types of tumors. GM-CSF is used as a treatment for humans to stimulate hematopoiesis and immune function, and to treat pulmonary alveolar proteinosis.

There are several environmental factors associated with the rising incidence of obesity and other body weight disorders, including an increasingly sedentary lifestyle and increased levels of caloric intake. Biochemical and metabolic differences between lean and obese individuals have been well-documented, and both deficient energy out-put as well as excessive caloric intake are functions of weight gain. The consumption of food, and resultant body weight, is highly regulated and appears to be linked to maintaining a constant level of adiposity. In humans, this is complicated by social factors, habits, daylight exposure and other more subtle factors. Fat stores appear to trigger hormonal messengers which regulate the subjective feelings of hunger and satiety. Control of feeding involves factors which emanate from both the central nervous system (CNS) and periphery, including the CNS generally, adrenals, gastrointestinal tract, and adipose tissue.

Selective breeding of mutant mice with chronic disorders of body weight led to the discovery of important genes involved in the modulation of food intake and long-term regulation of body weight. One of these is the ob gene. The ob gene product, leptin, is expressed in fat storage cells and acts as a circulating hormone signal that enters the brain an communicates the level of fat to the hypothalamic neurons. By means of this signal, body weight is kept reasonably constant over a broad range of activity and diet. Levels of circulating leptin correlate with fat mass. When plasma leptin falls below certain levels due to a decrease in fat mass, there are changes in energy expenditure and feeding behavior directed to restoration of the previous fat mass.

Methods are described herein for the modulation of food intake and bodyweight related activities, including, for example, treatment of metabolic and body weight disorders such as obesity, diabetes, and cachexia, and regulation of body weight and thermogenises. Accordingly, one embodiment of the present invention provides a method for modulating metabolic activity comprising administering to a subject an agent that acts via a GM-CSF signaling pathway in an amount effective for modulating metabolic activity. A more specific embodiment is directed to this method wherein the metabolic activity comprises food intake. Another embodiment of the invention is directed to a method for regulating body weight comprising administering to the subject an agent that acts via a GM-CSF signaling pathway an amount effective for regulating body weight. Other methods are directed to determining compounds which may be useful for regulating metabolic activity or body weight in a particular species by contacting the compound to a GM-CSF receptor and determining whether and to what extent it binds with the GM-CSF receptor.

Modulation comprises increasing or decreasing the activity from a homeostatic or initial level. Regulation comprises increasing or decreasing body weight from a homeostatic or initial level.

More specific embodiments of these methods contemplate GM-CSF, or any pharmacological agonist of GM-CSF, as the agent, while a further aspect contemplates a GM-CSF antagonist as the agent.

An agonist is defined as any substance which enhances or promotes the physiological action of a different substance. Generally, when acting on the receptor of another substance, an agonist produces a similar pharmacological effect. The effect does not have to be equal in magnitude, but should engender a qualitatively similar response, for example, excitatory or inhibitory. An agonist for purposes of a signaling pathway should act within that pathway to produce a qualitatively similar signal. Another embodiment contemplates modulation by use of a GM-CSF antagonist. An antagonist is any substance that reduces or blocks the physiological action of a different substance. In pharmacology, an antagonist is an agent that binds to the same site as would an active compound or agonist, and blocks the action of that agonist.

Since GM-CSF is known to cross the blood-brain barrier under some circumstances, peripheral administration, including but not limited to intradermal, intramuscular, intraperitoneal, subcutaneous, epidural, sublingual, intravaginal, transdermal, trans nasal and inhalation routes, impacts metabolic activity as well. Current clinical applications of GM-CSF to humans typically involve peripheral administration. For example, some typically reported treatments include 5-8 μg/kg/day either subcutaneously or inhaled as an aerosol for pulmonary disease, and 3-5 μg/kg/day subcutaneously for 14 days following irradiation or chemotherapy in the course of cancer treatment. Hence, some of the debilitating wasting experienced by many individuals with underlying immuno-involved conditions including, but not limited to, cancer, chronic inflammatory disease, rheumatoid or collagen disease, chronic infection and AIDs, may be caused or exacerbated by pro-immune system treatments. In addition, regardless of any peripheral to central transport of GM-CSF per se, the present invention contemplates methods comprising GM-CSF signaling agonists and antagonists which do cross the blood-brain barrier and impact the GM-CSF signaling pathways in the CNS via either single dosing or chronic administration.

The present invention provides methods directed to the regulation of metabolic activities in addition to food intake. Example 4, detailed below, illustrates that the observed change in body weight resulting from CNS administration of GM-CSF results from some factor in addition to decreased food intake. This indicates that GM-CSF stimulated some metabolic response other than that governing food intake alone.

Another embodiment of the invention is directed to methods of modulating metabolic activity and regulating body weight comprising manipulating a subject to express an agent that acts via a GM-CSF signaling pathway, in an amount effective for modulating metabolic activity and regulating body weight, respectively. A more specific embodiment is directed to these methods wherein the manipulation comprises or mimics an inflammatory event. The inventors realize that GM-CSF is a naturally occurring substance which is synthesized, expressed and endogenously released in response to certain stimuli. Given the expense associated with the manufacture of recombinant GM-CSF, there may be instances where it would be more cost effective to excite or inhibit GM-CSF signaling pathways by stimulating or inhibiting the body's own production. Since GM-CSF is expressed in response to inflammation, mimicking an inflammatory event would provide an additional means of modulating food intake and/or regulating body weight. Likewise, using genetic engineering methods already developed and well-known in the art, a subject can be prompted to express GM-CSF endogenously at enhanced rates or in enhanced amounts.

A further embodiment of the invention utilizes the GM-CSF receptor itself, under either in vivo or in vitro conditions, to identify compounds which may modulate or regulate metabolic activity or body weight in a species of therapeutic interest. These methods comprise bringing the test compound into contact with a GM-CSF receptor and determining whether the test compound binds to the receptor. The GM-CSF receptor may be of a wild or engineered type, and may be specific to the species of interest or any other species. Additionally, the GM-CSF receptor may exist in vitro under laboratory conditions, isolated or otherwise distinct from its typical physiological context, or in vivo under physiological conditions wherein the GM-CSF receptor is physiologically functional. In one aspect, the GM-CSF receptor is expressed on a eukaryotic cell. In certain aspects, the GM-CSF receptor has an amino acid sequence that is greater than 80% identical to the amino acid sequence of human GM-CSF receptor. In other aspects, the GM-CSF receptor has an amino acid sequence that is greater than 90% identical to the amino acid sequence of the human GM-CSF receptor. In a further aspect, the GM-CSF receptor has an amino acid sequence which corresponds to the amino acid sequence for GM-CSF receptors or receptor subtypes of any particular mammalian and/or vertebrate species which may be of therapeutic interest. Or, the GM-CSF receptor may exist in vivo in any such species, with such test-compound-receptor contact observed, mediated, and/or measured indirectly or clinically. The test compound may be administered to a subject in vivo via any of the means disclosed herein.

Determination of “binding” and what constitutes sufficient binding or saturation to define a requisite binding affinity may be by any method known or yet to be developed in the biochemical arts. What percent saturation or what duration of binding is deemed notable or sufficient may vary by specific application and can be easily determined by routine experimentation. When tested in vivo, a determination of binding may be indirect and based on observation and measurement of downstream events in the GM-CSF or interdependent signaling pathways. For example, human GM-CSF is known to induce an increase in reactive oxygen species (ROS) and uses ROS for some signaling functions. Triggering of GM-CSF is also known to induce specific phosphorylation events. The inflammatory response mediated by GM-CSF is known to act at least partly via activation of extracellular signal-regulated kinase and phosphoinositide kinase pathways. Stimulation of GM-CSF transcription by Ca²⁺ is mediated by the calmodulin-dependent phosphatase calcineurin, and the transcription factors NF-AT, AP-1, and NF-κB have been implicated in this activation. Hence, there are many indirect means known and determinable for measuring binding efficacy of test compounds to GM-CSF which allow in vivo identification of candidate compounds for regulating metabolic activity or body weight.

The inventive methods include embodiments which exploit the ability to measure these in vivo or functional GM-CSF downstream events. One embodiment specifically involves measuring a state of the GM-CSF receptor or a downstream state of events that are in a GM-CSF signaling pathway or a signaling pathway interdependent to it. A more specific embodiment relates to determining whether the test compound activates the GM-CSF receptor and involves measuring a phosphorylation state of the GM-CSF receptor or a phosphorylation state of events or cellular constituents that are in a GM-CSF signaling pathway or one interdependent to it. Another specific embodiment of the method for identifying candidate compounds involves determining whether the test compound activates the GM-CSF receptor by measuring enzymatic activity of the GM-CSF receptor or the enzymatic activity of downstream events or cellular constituents which are in the GM-CSF signaling pathway. In a further aspect, the cell expressing functional GM-CSF receptor further comprises a reporter gene operatively associated with activation of the GM-CSF receptor, and measuring expression of the reporter gene.

Compounds which bind to the GM-CSF receptor may be selected and further assessed for whether the test compound modulates metabolic activity or body weight in a model system and whether they are suitable candidate compounds for regulating metabolic activity or body weight in a species of therapeutic interest.

### EXAMPLE 1

This example illustrates both the methods for modulating the metabolic activity of food intake and for regulating body weight. Adult Long-Evans rats are divided into four groups, each receiving injections into the third cerebral ventrical (ic3v) of the brain: a control group (1) injected with vehicle only consisting of 2 μl 0.05% bovine serum albumin in saline, and 3 experimental groups injected with (2) 0.03, (3) 0.06 and (4) 0.6 μg recombinant rat GM-CSF in 2 μl BSA/saline. Rats are all injected at 9:00 am, at which point food is withdrawn. Food is made available at 1:00 pm, and food intake is measured at 3:00 pm, 5:00 pm and 9:00 am the following two days. Body weight is measured initially, and at the 24, 48 and 72 hour marks as well. 24-hour FI is measured to be 23 g, 18 g, 17 g and 15 g respectively for groups 1, 2, 3 and 4. 24-hour body weight change is measured to be −10 g, −13 g, −16 g and −20 g respectively for groups 1, 2, 3, and 4. Results have been consistent in multiple trials to date. Hence, it can be reliably stated that FI and body weights are consistently and reproducibly lower in rats injected with GM-CSF than in rats injected with vehicle, and that both are lower with increasing dosage.

### EXAMPLE 2

This example illustrates that the decreases in FI and body weight are not due to secondary effects of GM-CSF causing visceral illness. Two experimental techniques are employed to determine this: conditioned taste aversion (CTA) and sodium appetite tests. Both of these tests are well-known techniques in the biopharmacological arts for determining the presence of visceral disturbances, and for teasing that factor from results dependent upon measuring consumption. Essentially, the CTA involves comparing groups of rats who have been conditioned to find flavored water aversive by pairing the visceral illness triggered by LiCl consumption with exposure to this flavor, with groups of rats who received vehicle, and increasing doses of GM-CSF in association with consumption of flavored water. It is a well-known phenomena that if consumption of a novel food is paired with a nausea-inducing trigger, rats will subsequently avoid consumption of that food. Long-Evans rats are divided into 4 pre-conditioned groups: (1) receives IP LiCl treatment paired with flavored water, (2) receives IP saline treatment paired with flavored water, (3) receives ic3v vehicle paired with flavored water, and (4) receives ic3v GM-CSF paired with flavored water. Later, the groups receive their treatments, and are tested for any conditioned aversion to the flavored water by measuring their preference ratio after a 2 hour intake. The groups exhibit preference ratios of 0.1, 0.85, 0.65 and 0.65 respectively. This clearly indicates that the effects observed in example 1 are not due to secondary illness attributable to GM-CSF. Similar results and determinations are obtained from the Sodium Appetite Test.

### EXAMPLE 3

This example illustrates that the CNS GM-CSF signaling pathway involved in the FI and bodyweight reductions noted herein does not appear to be impacted by a single IP dose. In this case, a single intraperitoneal injection of 6 μg/kg GM-CSF is given to one group of Long Evans rats, while the second group receives IP saline. This is 10× the maximum ic3v dosage tested above, and does not result in reduced FI or body weight. This dosage is considered comparable to GM-CSF doses given in clinical applications, however, such clinical dosing is usually administered chronically. This result cannot necessarily be interpreted as indicating that GM-CSF peripheral pathways are not implicated, only that a single dose is not effective. GM-CSF has been found to cross the blood-brain barrier, though, apparently an effective amount does not cross when a single dose is administered. Clinical applications typically involve chronic administration, and transport across the blood brain barrier may require a saturation and/or receptor adaptation that occurs as a function of continual exposure over time. However, it does appear clear from this experiment that the GM-CSF signaling pathways implicated in food intake and body weight regulation are primarily localized in the CNS.

### EXAMPLE 4

This example illustrates that triggering the CNS GM-CSF signaling pathway impacts metabolic activity other than food intake. In this experiment, food is withheld for 24 hours after the treatment injections, and rats receiving ic3v GM-CSF still lose more body weight than rats receiving ic3v vehicle. After 24 hours post treatment fasting, the vehicle group demonstrates a weight change on average of −22 g, while the GM-CSF group demonstrates a an average weight change of −30 g. During an immediately subsequent 24 hour re-feeding regimen, the vehicle group demonstrates an average FI of 35 g, while the GM-CSF group demonstrates an average FI of 26 g. These results indicate that GM-CSF triggering impacts some factor related to energy expenditure, in addition to food intake. A further experiment is conducted in order to determine if body temperature is a factor being impacted. In this case, the rats receive the 9:00 am treatment injections, fast for 6 hours, at which point rectal body temperatures are determined. In two trials of this experiment, the results were inconclusive.

These experiments summarily indicate that (1) ic3v GM-CSF suppressed food intake and reduced body weight for as long as 72 hours, (2) GM-CSF did not support a conditioned taste aversion or loss of Na-appetite, (3) A single IP dose of 6 μg did not alter food intake or body weight, (4) weight loss in test rats was greater than in controls even when food-deprived, and (5) body temperature may or may not be increased.

The foregoing descriptions and examples of the various embodiments of the invention have been presented for the purposes of illustration and are not intended to be exhaustive or to limit the invention to the precise form disclosed. Many alternatives, modifications and variations will be apparent to those skilled in the art of neuropsychopharmacology. Accordingly, this invention is intended to embrace all alternatives, modifications and variations that have been discussed herein, and others that fall within the spirit and broad scope of the claims.

1. A method for modulating metabolic activity comprising administering to a subject an agent that acts via a GM-CSF signaling pathway in an amount effective for modulating metabolic activity.

2. The method recited in claim 1 wherein metabolic activity comprises food intake.

3. The method recited in claim 1 wherein the modulating comprises increasing metabolic activity.

4. The method recited in claim 1 wherein the modulating comprises decreasing metabolic activity.

5. The method recited in claim 1 wherein the agent comprises GM-CSF or a pharmacological agonist thereof.

6. The method recited in claim 1 wherein the agent comprises GM-CSF.

7. The method recited in claim 1 wherein the agent comprises a pharmacological antagonist of GM-CSF.

8. The method recited in claim 1 wherein the subject is human.

9. The method recited in claim 1 wherein the signaling pathway is in the Central Nervous System.

10. The method recited in claim 1 wherein the agent crosses the blood brain barrier.

11. The method recited claim 1 where the agent is administered by a/an intradermal, intramuscular, intraperitoneal, subcutaneous, epidural, sublingual, intracerebral, intraventricular, intravaginal, transdermal, intranasal or inhalation route.

12. The method recited in claim 11 wherein the route is intracerebral or intraventricular.

13. A method for modulating metabolic activity comprising manipulating a subject to express an agent in an amount effective to modulate metabolic activity wherein the agent comprises GM-CSF or acts via a GM-CSF signaling pathway.

14. The method recited in claim 13 wherein the manipulation comprises or mimics an inflammatory event.

15. The method recited in claim 13 wherein the manipulation is via genetic engineering.

16. A method for regulating body weight comprising administering to a subject an agent that acts via a signaling pathway of GM-CSF an amount effective for regulating the body weight.

17. The method recited in claim 16 wherein regulating comprises decreasing body weight.

18. The method recited in claim 16 wherein regulating comprises increasing body weight.

19. The method recited in claim 16 wherein the agent is GM-CSF or a pharmaceutical agonist thereof.

20. The method recited in claim 16 wherein the agent is GM-CSF.

21. The method recited in claim 16 wherein the agent is a pharmaceutical antagonist of GM-CSF.

22. The method recited in claim 16 wherein the subject is human.

23. The method recited in claim 16 wherein the subject has a bodyweight disorder.

24. The method recited in claim 23 wherein the disorder comprises obesity, anorexia nervosa, or chachexia.

25. The method recited in claim 16 wherein the subject is experiencing cancer, cancer treatment, chronic inflammatory disease, rheumatoid or collagen disease or chronic infection.

26. The method recited in claim 16 wherein the signaling pathway is in the Central Nervous System.

27. The method recited in claim 16 wherein the agent crosses the blood brain barrier.

28. The method recited claim 16 wherein the agent is administered by a/an intradermal, intramuscular, intraperitoneal, subcutaneous, epidural, sublingual, intracerebral, intraventricular, intravaginal, transdermal, intranasal or inhalation route.

29. The method recited in claim 28 wherein the route is intracerebral or intraventricular.

30. A method of regulating body weight comprising manipulating a subject to express an agent in an amount effective for regulating body weight wherein the agent acts via a GM-CSF signaling pathway.

31. The method recited in claim 30 wherein the manipulation comprises or mimics an inflammatory event.

32. The method recited in claim 31 wherein the manipulation is via genetic engineering.

33. A method for treatment of idiopathic body weight disorders comprising administering to a subject an agent that acts via a GM-CSF signaling pathway in an amount effective to treat the idiopathic body weight disorder.

34. A method for treatment of disease-related body weight disorders comprising administering to a subject an agent that acts via a GM-CSF signaling pathway in an amount effective to treat the disease-related body weight disorder.

35. A method for identifying candidate compounds for regulating metabolic activity or body weight, comprising;
a. contacting a test compound with a GM-CSF receptor;
b. determining whether the test compound binds to the GM-CSF receptor;
c. selecting those compounds that bind to GM-CSF receptor and further determining whether the test compound modulates metabolic activity or body weight in a model system that is capable of evaluating metabolic activity or body weight, and
d. identifying those test compounds that modulate metabolic activity or body weight in a model system as candidate compounds for regulating metabolic activity or body weight in a species of therapeutic interest.

36. The method for identifying candidate compounds according to claim 35 wherein the GM-CSF receptor has an amino acid sequence that is greater than 80% identical to a sequence of human GM-CSF receptor.

37. The method for identifying candidate compounds according to claim 35 wherein the GM-CSF receptor has an amino acid sequence that is greater than 90% identical to a sequence of human GM-CSF receptor.

38. The method for identifying candidate compounds according to claim 35 wherein the GM-CSF receptor has an amino acid sequence corresponding to the amino acid sequence of GM-GSF receptors or receptor subtypes for any mammalian or vertebrate species.

39. A method for identifying candidate compounds for regulating metabolic activity or body weight, comprising:
a. contacting a test compound with a cell expressing a functional GM-CSF receptor,
b. determining whether the test compound activates the GM-CSF receptor;
c. selecting those compounds that activate the GM-CSF receptor and further determining whether the test compound modulates metabolic activity or body weight in a model system that is capable of evaluating metabolic activity or body weight, and
d. identifying those test compounds that modulate metabolic activity or body weight as candidate compounds for regulating metabolic activity or body weight in a species of therapeutic interest.

40. The method for identifying candidate compounds according to claim 39 wherein the GM-CSF receptor has an amino acid sequence that is greater than 80% identical to a sequence of human GM-CSF receptor.

41. The method for identifying candidate compounds according to claim 39 wherein the GM-CSF receptor has an amino acid sequence that is greater than 90% identical to a sequence of human GM-CSF receptor.

42. The method for identifying candidate compounds according to claim 39 wherein the GM-CSF receptor has an amino acid sequence corresponding to the amino acid sequence of the species of therapeutic interest.

43. The method for identifying candidate compounds according to claim 39, in which the GM-CSF receptor is expressed on a eukaryotic cell.

44. The method for identifying candidate compounds according to claim 39, in which determining whether the test compound activates the GM-CSF receptor involves measuring a state of the GM-CSF receptor or a down-stream state of cellular constituents that are in a GM-CSF signaling pathway.

45. The method for identifying candidate compounds according to claim 39, in which determining whether the test compound activates the GM-CSF receptor involves measuring a phosphorylation state of the GM-CSF receptor or a phosphorylation state of cellular constituents that are in a GM-CSF signaling pathway.

46. The method for identifying candidate compounds according to claim 39, in which determining whether the test compound activates the GM-CSF receptor involves measuring enzymatic activity of the GM-CSF receptor or enzymatic activity of cellular constituents that are in a GM-CSF signaling pathway.

47. The method for identifying candidate compounds according to claim 39, in which the cell further comprises a reporter gene operatively associated with activation of the GM-CSF receptor, and measuring the expression of the reporter gene.
